(19) United States
(12) Patent Application Publication
(10) Pub. No.: US 2026/0354695 A1
(43) Pub. Date: Nov. 12, 2026

(54) Systems and Methods for Providing Smoothened Pressure

(71) Applicant: Becton, Dickinson and Company, Franklin Lakes, NJ (US)

(72) Inventor: Andrew Nguyen HOAN, Franklin Lakes, NJ (US)

(21) Appl. No.: 19/139,222

(22) PCT Filed: Dec. 18, 2023
(86) PCT No.: PCT/US2023/084600

§ 371 Date: Jun. 13, 2025

Related U.S. Application Data
(60) Provisional application No. 63/476,139, filed on Dec. 19, 2022.

(51) Int. Cl. A61B 5/0235; A61B 5/00; A61B 5/021; A61B 5/022

(52) U.S. Cl. CPC A61B 5/0235; A61B 5/02141; A61B 5/02241; A61B 5/6825

(57) ABSTRACT

Systems and methods for providing smoothened pressure are provided. Generally, a pressure isolation system can be utilized to isolate pressure from its source, resulting in mitigation of pulsations provided by the pressure source. The pressure isolation system can include two or more accumulators and set of multiway valves for controlling fluidic accumulation and distribution. Alternatively, pressure can be generated in a piston system utilizing a chamber, a piston and a set of multiway valves. The piston system can pull fluid to generate pressurized fluid that can be then be distributed.

### CROSS-REFERENCE TO RELATED APPLICATIONS

The current application claims priority under 35 U.S.C. 119(e) to U.S. Provisional Application No. 63/476,139, entitled “SYSTEMS AND METHODS FOR PROVIDING SMOOTHENED PRESSURE” to Hoan et al., filed Dec. 19, 2022, the disclosure of which is incorporated herein by reference in its entirety.

### TECHNOLOGICAL FIELD

The disclosure is generally directed to systems and methods for providing smoothened pressure, and more specifically for reducing pressure pulses for use in medical devices, such as a blood pressure monitoring system.

### BACKGROUND

Continuous noninvasive blood pressure monitors enable real-time measurement of blood pressure waves and derived hemodynamic parameters. Multiple techniques can be utilized including the volume clamp method.

Volume clamp method measures arterial blood pressure at an extremity (e.g., finger) utilizing an inflatable pressure cuff, a light source (e.g., light emitting diode (LED)), and light sensor. The pressure in the cuff is adjusted to keep the diameter of the artery constant (the unloaded state), in which the diameter is determined via the light source and light sensor. The pressure within the inflatable cuff represents the arterial pressure of the finger artery. A pressure pump supplies the pressure to the inflatable cuff.

### SUMMARY

Various systems and methods for providing smoothened pressure utilize a pressure isolating system that comprises a set of two or more accumulator chambers. The two or more accumulator chambers are configured to accumulate and release pressure such that the released pressure is isolated from the pressure source and provided continuously. Various systems and methods for providing smoothened pressure utilize a piston system that comprises a chamber bifurcated by a piston. The two sides of the bifurcated chamber are programmed to accumulate and release fluid such that the release of fluid is smoothened and continuous.

In an aspect, a system is for isolating pressure. The system comprises a set of two or more accumulator chambers capable of being in fluidic connection with a pressure supply. The system further comprises an inlet multiway valve. The inlet multiway valve has an inlet port capable of being in fluidic connection with the pressure supply and an outlet port for each accumulator chamber that is in fluidic connection with its accumulator chamber. The system further comprises an outlet multiway valve. The outlet multiway valve has an inlet port for each accumulator chamber that is in fluidic connection with its accumulator chamber.

In some implementations, the inlet multiway valve and the outlet multiway valve are programmed to sequentially alternate accumulation and release of pressure among the set of two or more accumulator chambers.

In some implementations, the inlet multiway valve and the outlet multiway valve are programmed via a timing mechanism.

In some implementations, the inlet multiway valve and the outlet multiway valve are programmed via a pressure sensor.

In some implementations, the set of two or more accumulator chambers includes a first accumulator chamber and a second accumulator chamber. The sequentially alternate accumulation and release of pressure among the set of two or more accumulator chambers comprises: when the first accumulator chamber accumulates pressure from the pressure source via the inlet multiway valve, the second accumulator chamber concurrently releases pressure via the outlet multiway valve; and when the second accumulator chamber accumulates pressure from the pressure source via the inlet multiway valve, the first accumulator chamber concurrently releases pressure via the outlet multiway valve.

In some implementations, when a first accumulator of the set of two or more accumulator chambers receives pressure from the pressure supply, the multiway inlet valve is open and the multiway outlet valve is closed. When the first accumulator releases pressure the multiway inlet valve is closed and the multiway outlet valve is open.

In some implementations, the system further comprises the pressure supply. The pressure supply in fluidic connection with the set of two or more accumulator chambers via the inlet multiway valve that is between the pressure supply and the set of two or more accumulator chambers.

In some implementations, the pressure supply is a pressure pump.

In some implementations, the pressure pump is a positive-displacement pump, a centrifugal pump, or an axial-flow pump.

In some implementations, the pressure pump is a rotary-type pump, a reciprocating-type pump, a linear-type pump, a piezoelectric pump, or a pneumatic pump.

In some implementations, the inlet multiway valve or the outlet multiway valve is: a solenoid valve, a hydraulic valve, a piezoelectric valve, or a motorized valve.

In some implementations, the system further comprises a device in fluidic connection with multiway outflow valve and configured to receive pressure from the set of two or more accumulator chambers.

In some implementations, the device is a medical device.

In some implementations, the device is blood pressure monitoring system.

In some implementations, the blood pressure monitoring system comprises a pressure cuff configured to receive pressure from the set of two or more accumulator chambers.

In some implementations, the blood pressure monitoring system utilizes a volume clamp method to measure blood pressure.

In some implementations, a process is for continuously providing isolating pressure from a pressure source. The process provides a system for isolating pressure. The system comprises a set of two or more accumulator chambers in fluidic connection with a pressure supply. The set of two or more accumulator chambers includes a first accumulator chamber and second accumulator chamber. The system further comprises an inlet multiway valve. The inlet multiway valve has an inlet port in fluidic connection with the pressure supply and an outlet port for each accumulator chamber that is in fluidic connection with its accumulator chamber. The system further comprises an outlet multiway valve. The outlet multiway valve has an inlet port for each accumulator chamber that is in fluidic connection with its accumulator chamber. The process further accumulates pressure from the pressure supply through the inlet multiway valve into the first accumulator chamber while simultaneously releasing pressure from the second accumulator chamber through the outlet multiway valve. The inlet port of the outlet multiway valve in fluidic connection with the first accumulator chamber is closed and the outlet port of the inlet multiway valve in fluidic connection with the second accumulator chamber is closed. The process further releases pressure from the first accumulator chamber through the outlet multiway valve while simultaneously accumulating pressure from the pressure supply through the inlet multiway valve into the second accumulator chamber. The inlet port of the outlet multiway valve in fluidic connection with the second accumulator chamber is closed and the outlet port of the inlet multiway valve in fluidic connection with the first accumulator chamber is closed.

In some implementations, a system is for providing smoothened pressure. The system comprises a chamber bifurcated by a piston, yielding a first side and second side of the chamber, wherein the piston provides a seal to isolate the two sides of the chamber. The piston is powered to move back and forth within the chamber. The system further comprises a first multiway valve in fluidic connection with the first side of the chamber. The first multiway valve is configured to receive fluid and transfer the fluid into the first side of the chamber, and wherein the first multiway valve is further configured to release the fluid from the first side of the chamber. The system further comprises a second multiway valve in fluidic connection with the second side of the chamber. The second multiway valve is configured to receive fluid and transfer the fluid into the second side of the chamber, and wherein the second multiway valve is further configured to release the fluid from the first side of the chamber.

In some implementations, the piston is powered to move back and forth within the chamber by a motorized crank with a lever that is in connection with the piston.

In some implementations, when the piston is powered to move away from the first side of the chamber and towards the second side chamber, the first multiway valve allows fluid to accumulate within the first side of the chamber and the second valve simultaneously allows fluid to be released from the second side of the chamber.

In some implementations, the first multiway valve and the second multiway valve are programmed to provide continuous pressure released from the chamber.

In some implementations, the first multiway valve and the second multiway valve work in conjunction with the movement of the piston within the chamber to provide the continuous pressure.

In some implementations, the first multiway valve and the second multiway valve are programmed via a timing mechanism.

In some implementations, the first multiway valve and the second multiway valve are programmed via a pressure sensor.

In some implementations, the inlet multiway valve or the outlet multiway valve is: a solenoid valve, a hydraulic valve, a piezoelectric valve, or a motorized valve.

In some implementations, the system further comprises a device in fluidic connection with the first multiway valve or the second multiway valve and configured to receive pressure from the chamber.

In some implementations, the system further comprises a device in fluidic connection with the first multiway valve and the second multiway valve and configured to receive pressure from the chamber.

In some implementations, the device is a medical device.

In some implementations, the device is blood pressure monitoring system.

In some implementations, the blood pressure monitoring system comprises a pressure cuff configured to receive pressure from the chamber.

In some implementations, the blood pressure monitoring system utilizes a volume clamp method to measure blood pressure.

In some implementations, a process is for continuously providing smoothened pressure from a pressure source. The process provides a piston system. The system comprises a chamber bifurcated by a piston, yielding a first side and second side of the chamber. The piston provides a seal to isolate the two sides of the chamber, and wherein the piston is powered to move back and forth within the chamber. The system further comprises a first multiway valve in fluidic connection with the first side of the chamber. The first multiway valve is configured to receive fluid and transfer the fluid into the first side of the chamber. The first multiway valve is further configured to release the fluid from the first side of the chamber. The system further comprises a second multiway valve in fluidic connection with the second side of the chamber. The second multiway valve is configured to receive fluid and transfer the fluid into the second side of the chamber. The second multiway valve is further configured to release the fluid from the first side of the chamber. The process further accumulates fluid within the first side of the chamber via the first multiway valve while simultaneously releasing fluid from the second side of the chamber via the second multiway valve. The accumulation of fluid in the first side of the chamber and the release of fluid from the second side of the chamber is induced by moving the piston away from the first side of the chamber and towards the second side of the chamber. The process releases fluid from the first side of the chamber via the first multiway valve while simultaneously accumulating fluid within the second side of the chamber via the second multiway valve. The release of fluid in the first side of the chamber and the accumulation of fluid from the second side of the chamber is induced by moving the piston toward from the first side of the chamber and away the second side of the chamber.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1 provides a mock example of data graph of pressure showing the pressure pulsation as generated by a pressure source.

FIG. 2 provides a mock example of data graph of pressure showing smoothened pressure when isolated from a pressure source.

FIG. 3 provides an example of a pressure isolation system.

FIG. 4 provides an example of a process using a pressure isolation system.

FIG. 5 provides an example of a piston system for providing smoothened pressure.

FIG. 6 provides an example of a process using a piston system for providing smoothened pressure.

FIG. 7 provides an example of a blood pressure monitoring system utilizing a pressure supply.

FIG. 8 provides an example of a blood pressure cuff fitted onto a finger for performing continuous blood pressure monitoring.

### DETAILED DESCRIPTION

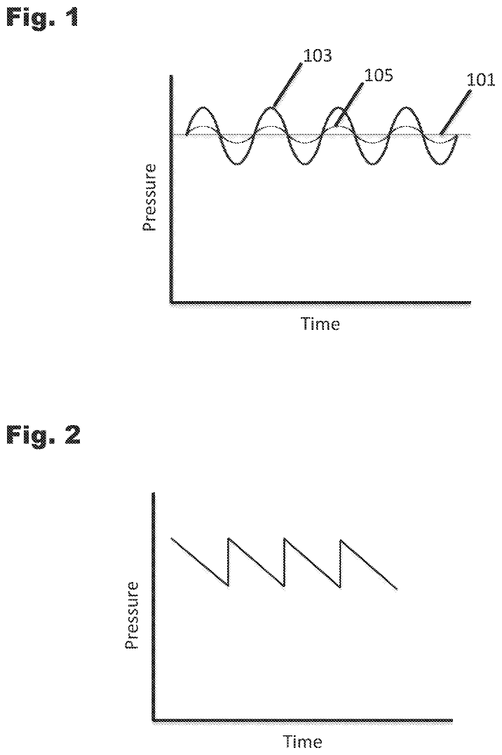

The current disclosure details systems and methods for providing smoothened pressure, which can be utilized within a blood pressure monitoring system or other medical devices that utilize a pressure source. Many pressure pumps and other pressure sources typically provide pulsating pressure, and although the pulsation is minor, it can cause inaccuracies in sensitive measurements and treatments. For example, a pneumatic diaphragm pump creates pressure by utilizing flexible diaphragms that move back and forth, resulting in pulsation of the pressure generated. FIG. 1 provides a mock example of a data graph of a pressure measurement that is typical of a pressure source providing a constant pressure. While the average pressure 101 provided is constant, that actual provided pressure 103 pulsates up and down, yielding a pulsation effect of the average provided pressure. The pulsation effect can impact machinery (especially medical devices) that are sensitive to pressure pulsations. For example, pulsations of pressure provided for a pressure cuff utilized within a continuous blood pressure system can harm the accuracy of the blood pressure readings.

Prior art techniques to reduce pressure pulsations within medical devices typically rely on expansion chambers. Generally, pressure is generated by the source pump and then passed through one or more expansion chambers prior to being utilized by the medical device. The expansion chambers allow the pressure to expand therein as it passes therethrough, resulting in a damping effect on the pressure pulsations. Referring back to FIG. 1, pressure curve 105 is an example of a pressure provided that has passed through expansion chambers, which dampens the pulsations of pressure as compared to pressure curve 105. Although the pulsations are dampened, the pulsations can still cause disruptions in sensitive applications.

It is a goal of the current application to further reduce or even eliminate the pulsation effect by various means. In one implementation, pressure is isolated from its source. Instead of just passing pressure through an expansion chamber, a pressure isolation system can comprise a means for collecting the pressure from the source within an isolated accumulator chamber, cut-off connection with the pressure source, and then distribute the pressure from the chamber. The smoothened pressure can yield better accuracy when used in a blood pressure monitoring system or other medical device that benefits from less pressure pulsation. FIG. 2 provides a mock example of a data graph of a pressure measurement in which the pressure has been collected and released from an isolation chamber. The pressure wanes each time it is released from an isolation pressure, but the pressure does not pulsate as it has been collected and isolated from the pressure source.

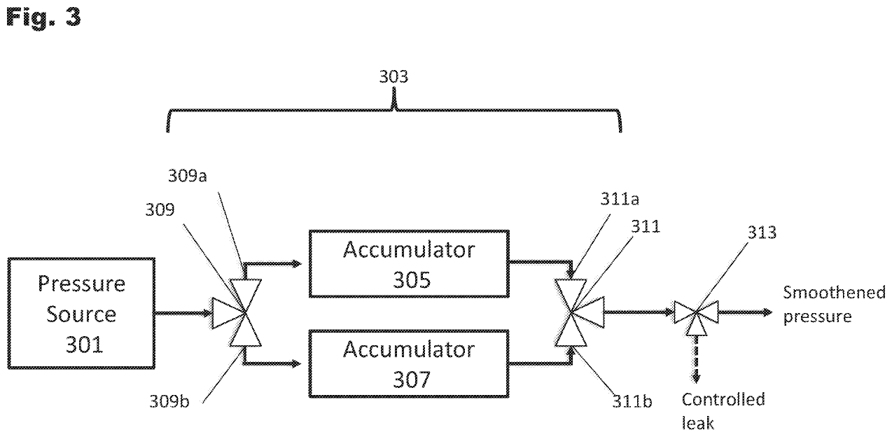

Provided in FIG. 3 is an overview system diagram of an example of a pressure isolation system. The pressure system comprises a pressure supply 301 and a pressure isolation system 303. Pressure supply 301 can be any source that can provide pressure, such a pressure pump. The pressure pump can be a positive-displacement pump, a centrifugal pump, an axial-flow pump or any other pump capable of generating pressure. In some implementations, the pressure pump is a positive-displacement pump. Types of positive-displacement pumps that can be utilized include (but are not limited to) rotary-type pumps, reciprocating-type pumps, linear-type pumps, piezoelectric pumps, and pneumatic pumps. The various pumps each provide a relative amount of pressure pulsation as the pump mechanics facilitate the movement of fluid (liquid or gas).

The pressure provided by the pressure supply 301 is transferred to the pressure isolation system 303, which isolates pressure from the source to reduce or eliminate the pulsation of the pressure as provided by the pressure supply. A pressure isolation system can comprise a set of two or more accumulators and set of two or more multiway valves. A first multiway valve is an inlet valve for receiving pressure into the pressure isolation system, with an inlet port to the pressure supply and an outlet port to each of the two or more accumulators. A second multiway valve is an outlet valve for releasing the pressure from the pressure isolation system, with an inlet port from each of the two or more accumulators and an outlet port that releases smoothened pressure. The outlet port can be in connection with machinery or other devices, especially devices that are sensitive to pulsations in pressure.

As depicted in the example, pressure isolation system 303 comprises a first accumulator chamber 305 and a second accumulator chamber 307 with an inlet 3-way valve 309. Accumulator 305 and accumulator 307 are in fluidic connection with pressure supply 301 via inlet valve 309 therebetween. The 3-way valve can be any type of 3-way valve capable of controlling flow through the three ports. Nonlimiting examples of valves that can be utilized include solenoid valves, hydraulic valves, and motorized valves. 3-way valve 309 has an inlet port in fluidic connection with pressure supply 301 for receiving pressure generated by the pump. Valve 309 also has two outflow ports, a first outlet port 309a in fluidic connect with accumulator 305 and second outlet port 309b in fluidic connection with accumulator 307. Accumulator 305 and accumulator 307 are each also in fluidic connection with an outlet 3-way port 311, which receives the pressure from accumulator 305 via inlet port 311a and the pressure from accumulator 307 via inlet port 311b. An optional controlled leak 313 can be provided after the outlet 3-way port for controlling the pressure output.

To isolate the pressure from the pressure supply, each accumulator can release pressure independent of receiving the pressure from the pressure supply. To keep pressure continuous, the accumulators can be utilized in a sequential manner. A first accumulator can be receiving pressure from the pressure supply while the second accumulator is releasing pressure from its chamber. Once the second accumulator finishes releasing pressure, it begins receiving pressure as the first accumulator stops receiving pressure and begins releasing its stored pressure. By alternating the accumulation and release of pressure from the two accumulators, the released pressure will resemble pressure measurement as exemplified in FIG. 2, where the initial release of pressure from the first accumulator is higher and wanes until switched to the second accumulator, resulting in the sequential step pattern as shown. It is noted that although the release pressure has a step pattern, the pulsation effect is mitigated due to the isolation technique.

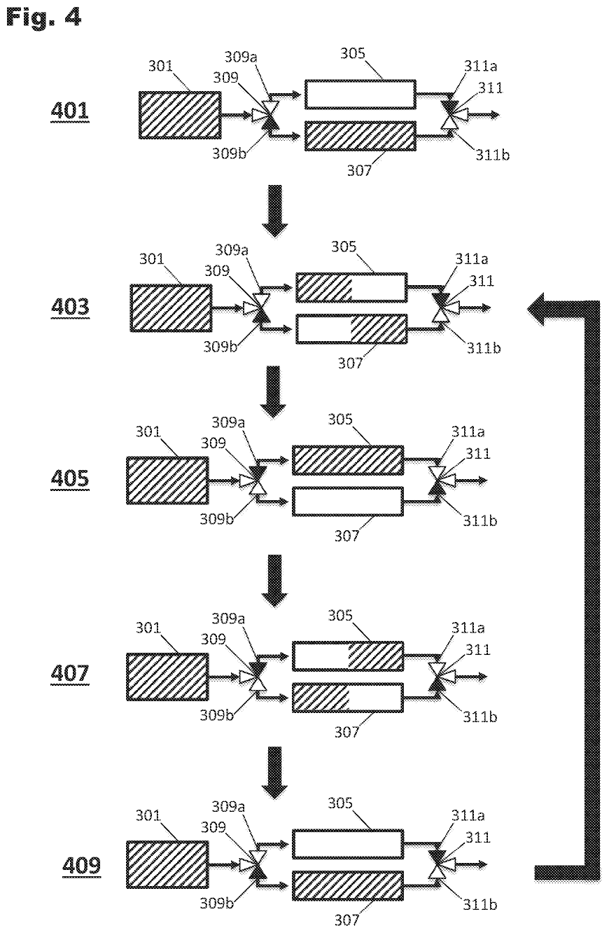

FIG. 4 provides an example of a method of isolating pressure using the pressure isolation system of FIG. 3. Within this figure, a hatch pattern indicates a pressure amount and a shaded valve port indicates a closed port whereas an unshaded valve port indicates an open port. As shown at step 401, accumulator 307 is full of pressure (which can be initially provided by the pressure source 301) and accumulator 305 is devoid of pressure. The pressure within accumulator 307 is isolated from pressure source 301 by closing valve port 309b. Further, the pressure within accumulator 307 is released out via valve 311 by opening valve 311b. As the pressure within accumulator 307 is being released, the pressure source 301 fills accumulator 305 via valve 309 by opening up valve port 309a. To accumulate the pressure within accumulator 305, valve port 311a is closed. Step 403 shows the halfway point of the accumulation of pressure within accumulator 305 and the release of pressure from accumulator 307. To maintain accumulation from the pressure source 301, valve port 309a remains open while valve port 311a remains closed. The pressure being released from accumulator 307 is isolated from pressure source 301 due to the valve port 309b being closed. Thus, the pressure released through valve port 311b from accumulator 307 has severely dampened or eliminated pulsations of pressure as provided by pressure source 301.

At step 405, pressure within accumulator 307 has been released and the pressure with accumulator 305 has been filled. At this moment, valve ports 309a and 311b are closed and valve ports 309b and 311a are opened. The closing and opening of these valve ports releases of pressure from accumulator 305 and accumulates pressure within accumulator 307. Step 407 shows the halfway point of the accumulation of pressure within accumulator 307 and the release of pressure from accumulator 305. To maintain accumulation from the pressure source 301, valve port 309b remains open while valve port 311b remains closed. The pressure being released from accumulator 305 is isolated from pressure source 301 due to the valve port 309a being closed. Thus, the pressure released through valve port 311a from accumulator 305 has severely dampened or eliminated pulsations of pressure as provided by pressure source 301.

At step 409, the pressure within accumulator 305 has been released and the pressure with accumulator 307 has been filled. At this moment, valve ports 309b and 311a are closed and valve ports 309a and 311b are opened, and the process is reinitiated. The process can continue indefinitely, ensuring continuous pressure without pulsation. The continuous outflow pressure can be utilized in medical machinery or other devices that benefit from smoothened pressure.

In some implementations, the outflow pressure is utilized in a blood pressure monitoring device. Generally, the smoothened pressure outflow travels through a pressure control system that senses the pressure amount and can adjust the outflow pressure as necessary (e.g., via a controlled pressure leak). The pressure is then transferred into a blood pressure cuff, which can be a cuff surrounding any extremity of a patient, such as (for example) an arm or a digit. In some implementations, the blood pressure monitoring system utilizes a volume clamp method for continuous blood pressure monitoring and thus the pressure is adjusted based on the amount of pressure to keep the diameter of a patient's artery constant via the pressure cuff. When a volume clamp method is utilized, the supplied pressure to the cuff that keeps the artery diameter constant is the blood pressure within that artery.

The multiway valves can be controlled and/or programmed in any way that allows for sequential alternation of accumulation and release of pressure within the set of accumulator chambers. In some implementations, the valves are on a timing mechanism such that the ports of the inlet and outlet multiway valves open and close based on periodic intervals. When the end of the periodic interval is reached, the inlet multiway valve will alternate the flow of pressure from the pressure source by terminating flow into a first accumulator into and initiating flow into a second accumulator. Concurrently, the outlet multiway valve will initiate the release of pressure from the first accumulator and terminate the release of pressure from the second accumulator. In some implementations, the valves rely a pressure sensor capable of sensing the amount of pressure within an accumulator or sensing the amount of pressure released. When the pressure within an accumulator or the amount of pressure released reaches a requisite pressure level, the inlet multiway valve will alternate the flow of pressure from the pressure source by terminating flow into a first accumulator into and initiating flow into a second accumulator. Concurrently, the outlet multiway valve will initiate the release of pressure from the first accumulator and terminate the release of pressure from the second accumulator.

While an exemplary pressure isolation system is described above with reference to FIGS. 3 and 4, it should be readily appreciated that a pressure isolation system can be implemented in any of a variety of ways including various combinations of accumulators and types of multiway valves. Furthermore, the housing for a pressure isolation system can take any form that allows for fluidic connection from the inlet valve to the set of accumulators to the outlet valve. Accordingly, a pressure isolation system should be understood as not limited to the specific pressure isolation system shown, the number of actuators, or the specific types of valves. Instead, a pressure isolation system can be implemented in a variety of ways as long the means for providing isolated pressure are included.

A variety of systems can be utilized to produce smoothened pressure. In one implementation, a powered piston and dual-sided chamber can be utilized. Generally, a piston can be utilized within a chamber. The piston can be powered to move back and forth within the chamber. As the piston is pulled away from one side, and in conjunction with a multiway valve, pressure can be built within that side. When the piston is pushed back to that side, it can smoothly push the pressure out such that it does not pulsate.

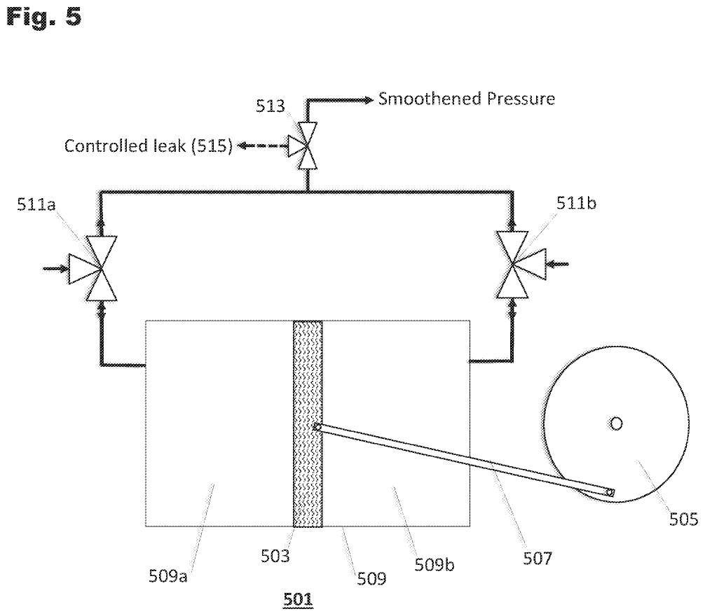

Provided in FIG. 5 is an exemplary piston system 501 for providing smoothened pressure. Piston system 501 comprises a piston 503 that is controlled by a motorized crank 505 and lever 507. Crank 505 can rotate about an axis, pulling and pushing piston 503 back and forth via lever 507. Piston 503 is provided within a chamber 509 that is bifurcated by piston 503. Piston 503 provides a seal between the two chamber sides (509a and 509b) such that pressure on one side cannot leak into the other side. Although a motorized crank is shown to power the piston, any means for powering a piston can be utilized.

Each chamber side (509a and 509b) is connected with its own multiway valve (511a and 511b). As shown, the multiway valves (511a and 511b) are 3-way valves, which are capable of bringing in a fluid (e.g., air) and distributing the air into chamber 509. Multiway valves (511a and 511b) are also capable of receiving smoothened pressure from chamber 509 to distribute as needed. The 3-way valve can be any type of 3-way valve capable of controlling flow through the three ports. Nonlimiting examples of valves that can be utilized include solenoid valves, hydraulic valves, and motorized valves.

Piston 503 can move back forth within the chamber, pulling fluid into the chamber, and then pushing pressurized fluid out of the chamber. Chamber 509 is in fluidic connection with the smoothened pressure outlet 513. An optional controlled leak 515 can be provided therebetween for controlling the pressure output. The outlet port 513 can be in connection with machinery or other devices, especially devices that are sensitive to pulsations in pressure.

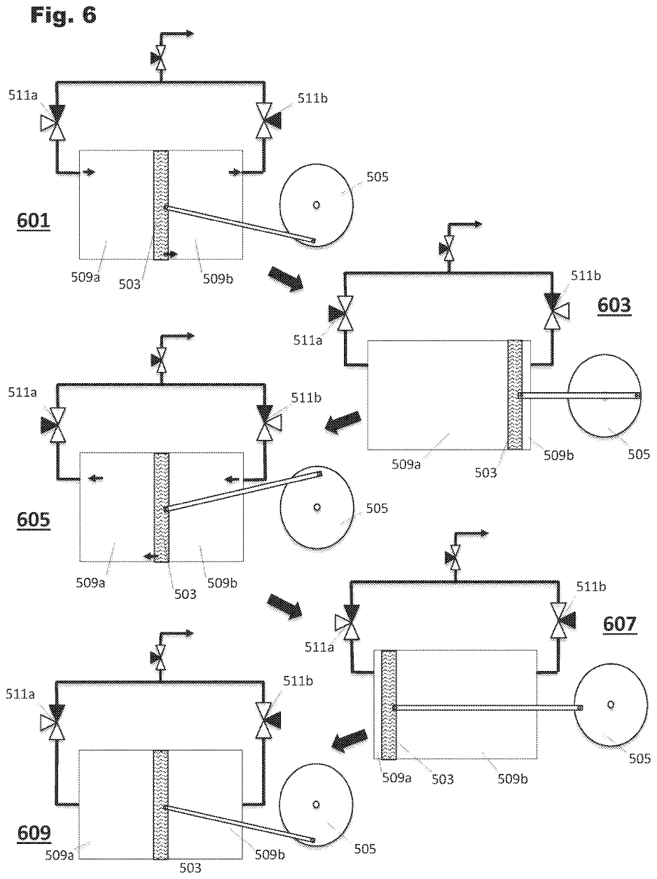

FIG. 6 provides an example of a method of providing smoothened pressure using the piston system of FIG. 5. Within this figure, a shaded valve port indicates a closed port whereas an unshaded valve port indicates an open port. As shown in step 601, piston 503 is in the center of chamber 509, indicating there is equal pressure in the two chamber sides (509a and 509b). Crank 505 is turning counter clockwise, resulting in piston 503 moving in a direction consistent with crank turn (e.g., as shown in the rightward direction). The movement of the piston the first direction results in fluid being pulled into the chamber via multiway valve 511a and pressurized fluid being pushed out of the chamber via multiway valve 511b.

At step 603, piston 503 reaches a stop in the first direction. This stop ends the pulling of fluid into the chamber via multiway valve 511a and the distribution of pressurized fluid via multiway valve 511b. Accordingly, multiway valve 511b closes its port for distributing smoothened pressure and opens its port of drawing in fluid and multiway valve 511a opens its port for distributing smoothened pressure and closes its port of drawing in fluid.

At step 605, crank 505 continues to turn counterclockwise resulting in piston 503 moving in a direction consistent with crank turn (e.g., as shown in the leftward direction). The movement of the piston the second direction results in fluid being pulled into the chamber via multiway valve 511b and pressurized fluid being pushed out of the chamber via multiway valve 511a.

At step 607, piston 503 reaches a stop in the second direction. This stop ends the pulling of fluid into the chamber via multiway valve 511b and the distribution of pressurized fluid via multiway valve 511a. Accordingly, multiway valve 511a closes its port for distributing smoothened pressure and opens its port of drawing in fluid and multiway valve 511b opens its port for distributing smoothened pressure and closes its port of drawing in fluid. Step 609 returns piston 503 and crank 505 to their position within step 601, such that the steps 601 to 609 can be repeated. The process can continue indefinitely, ensuring continuous pressure without pulsation. The continuous outflow pressure can be utilized in medical machinery or other devices that benefit from smoothened pressure.

In some implementations, the outflow pressure is utilized in a blood pressure monitoring device. Generally, the smoothened pressure outflow travels through a pressure control system that senses the pressure amount and can adjust the outflow pressure as necessary (e.g., via the controlled leak). The pressure is then transferred into a blood pressure cuff, which can be a cuff surrounding any extremity of a patient, such as (for example) an arm or a digit. In some implementations, the blood pressure monitoring system utilizes a volume clamp method for continuous blood pressure monitoring and thus the pressure is adjusted based on the amount of pressure to keep the diameter of a patient's artery constant via the pressure cuff. When a volume clamp method is utilized, the supplied pressure to the cuff that keeps the artery diameter constant is the blood pressure within that artery.

The multiway valves can be controlled and/or programmed in any way that allows for continuous release of pressure from the chamber by alternating the release of fluid between the first side and the second side. In some implementations, the valves are on a timing mechanism such that the ports of the inlet and outlet multiway valves open and close based on periodic intervals. When the end of the periodic interval is reached, the first multiway valve will alter the flow of fluid within the first side of the chamber by terminating fluid flow into the first side and initiating release of fluid from the first side. Concurrently, the second multiway valve will alter the flow of fluid within the second side of the chamber by terminating the release of fluid from the second side and initiating fluid flow into the second side. In some implementations, the valves rely a pressure sensor capable of sensing the amount of pressure within an accumulator. When the pressure within an accumulator reaches a requisite pressure level, the first multiway valve will alter the flow of fluid within the first side of the chamber by terminating fluid flow into the first side and initiating release of fluid from the first side. Concurrently, the second multiway valve will alter the flow of fluid within the second side of the chamber by terminating the release of fluid from the second side and initiating fluid flow into the second side.

While an example of a piston system for providing smoothened pressure is described above with reference to FIGS. 5 and 6, it should be readily appreciated that a pressure isolation system can be implemented in any of a variety of ways including various designs of chambers and pistons, various means for driving the piston, and various types of multiway valves. Furthermore, the housing for a piston system can take any form that allows for controlled fluidic connection and that ensures separation of fluid between the two sides of the chamber. Accordingly, a piston system should be understood as not limited to the specific piston system shown, the means for driving the piston, or the specific types of valves. Instead, a piston system can be implemented in a variety of ways as long the means for providing smoothened pressure are included.

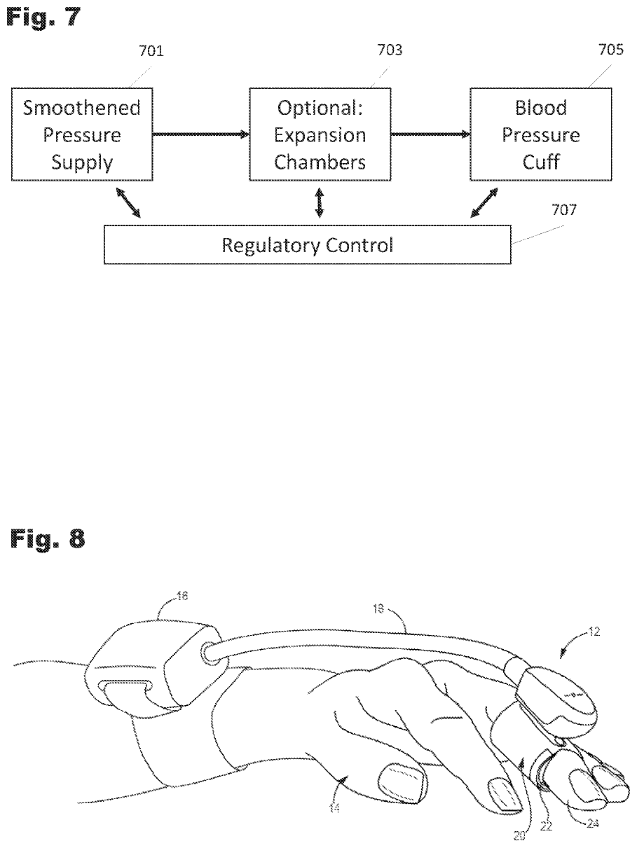

Provided in FIG. 7 is an overview system diagram of a pressure system utilized within a blood pressure monitoring system. The pressure system comprises a smoothened pressure supply 701, an optional set of expansion chambers 703, blood pressure cuff 705, and a regulatory control 707. Smoothened pressure supply 701 can be any source that can provide smoothened pressure, such the systems described in reference to FIGS. 3 to 6. In some implementations, the smoothened pressure supply comprises a pump for generating pressure and a set of two or more accumulators. In some implementations, the smoothened pressure supply comprises a piston that bifurcates a chamber, the piston capable of drawing and releasing pressure in and out the chamber.

The pressure provided by the pressure supply 701 is optionally transferred through expansion chambers 703 to further reduce pulsation of the pressure as provided by the supply. Expansion chambers 703 can comprise a set of one or more chambers that allow fluid to expand therein, resulting in damping of pressure pulsation. Each expansion chamber is in fluidic connection such that the fluid transfers sequentially therethrough. Generally, the smoothened and optionally dampened pressure outflow travels through a pressure control system that comprises a pressure sensor to sense the amount of pressure and can adjust the supplied pressure as necessary via regulatory control 707. The pressure is then transferred into a blood pressure cuff 705, which can be a cuff surrounding any extremity of a patient, such as (for example) an arm or a digit. In some implementations, the blood pressure monitoring system utilizes a volume clamp method for continuous blood pressure monitoring and thus the pressure is adjusted based on the amount of pressure to keep the diameter of a patient's artery constant via the pressure cuff. When a volume clamp method is utilized, the supplied pressure to the cuff that keeps the artery diameter constant is the blood pressure within that artery.

FIG. 8 provides a simplified perspective view a non-invasive hemodynamic sensor system 12 attached to hand 14. Although the sensor is depicted to be secured to a finger, it should be understood that volume clamp blood pressure measure and arterial pulsation can be performed on any body appendage capable of being receiving a blood pressure cuff. Examples of body appendages that can be utilized include (but are not limited to) an arm, a finger, a thumb, a wrist, an ankle, a leg, a toc, an car, a temple, etc.

Non-invasive hemodynamic sensor system 12 is capable of generating arterial blood pressure measurements through volume clamping. Sensor system 12 can include housing 16, connector 18, cuff 20, and pressurizable bladder 22. In the illustrated example, cuff 20 is a ring or similar structure surrounding or bracketing finger 24 of hand 14, while housing 16 is a wrist-mounted device coupled to cuff 20 via connector 18. In the most general case, however, sensor system 12 can differ substantially from the layout illustrated in FIG. 8. Sensor system 12 can, for example, include multiple separate connectors 18 between elements attached to finger 24 (e.g. cuff 20), and/or can relocate housing 16 to other locations (e.g. integrated with cuff 20, or separately disposed at a peripheral location). Further, cuff 20 can be configured to fit on a variety of body appendages, including an arm, a finger, a thumb, a wrist, an ankle, a leg, a toe, an ear, a temple, etc. In the illustrated example, cuff 20 surrounds a sensing region of a finger 24 of hand 14. At least one artery passes through the sensing region, and generally other tissue may be present (e.g., muscle, skin, connective tissue, vein, capillaries). Cuff 20 also anchors pressurizable bladder 22, which can for example be an expandable annular fluid bladder fed by a fluid line included within connector 18, or from another source, which can be provided by, for example, pressure supplies as described in association with FIGS. 3 to 6. Generally, fluid utilized to inflate a bladder can be air. In the most general case, however, pressurizable bladder 22 can be any sort of mechanism suited to apply pressure to finger 24.

### EXAMPLES

Example 1. A system for isolating pressure, comprising:
a set of two or more accumulator chambers capable of being in fluidic connection with a pressure supply;
an inlet multiway valve, wherein the inlet multiway valve has an inlet port capable of being in fluidic connection with the pressure supply and an outlet port for each accumulator chamber that is in fluidic connection with its accumulator chamber; and
an outlet multiway valve, wherein the outlet multiway valve has an inlet port for each accumulator chamber that is in fluidic connection with its accumulator chamber.

Example 2. The system of example 1, wherein the inlet multiway valve and the outlet multiway valve are programmed to sequentially alternate accumulation and release of pressure among the set of two or more accumulator chambers.

Example 3. The system of example 2, wherein the inlet multiway valve and the outlet multiway valve are programmed via a timing mechanism.

Example 4. The system of example 2, wherein the inlet multiway valve and the outlet multiway valve are programmed via a pressure sensor.

Example 5. The system of example 2, 3, or 4, wherein the set of two or more accumulator chambers includes a first accumulator chamber and a second accumulator chamber; wherein the sequentially alternate accumulation and release of pressure among the set of two or more accumulator chambers comprises: when the first accumulator chamber accumulates pressure from the pressure source via the inlet multiway valve, the second accumulator chamber concurrently releases pressure via the outlet multiway valve; and when the second accumulator chamber accumulates pressure from the pressure source via the inlet multiway valve, the first accumulator chamber concurrently releases pressure via the outlet multiway valve.

Example 6. The system of any one of examples 1 to 5, wherein when a first accumulator of the set of two or more accumulator chambers receives pressure from the pressure supply, the multiway inlet valve is open and the multiway outlet valve is closed; and when the first accumulator releases pressure the multiway inlet valve is closed and the multiway outlet valve is open.

Example 7. The system of any one of examples 1 to 6 further comprising the pressure supply; wherein the pressure supply in fluidic connection with the set of two or more accumulator chambers via the inlet multiway valve that is between the pressure supply and the set of two or more accumulator chambers.

Example 8. The system of example 7, wherein the pressure supply is a pressure pump.

Example 9. The system of example 8, wherein the pressure pump is a positive-displacement pump, a centrifugal pump, or an axial-flow pump.

Example 10. The system of example 8, wherein the pressure pump is a rotary-type pump, a reciprocating-type pump, a linear-type pump, a piezoelectric pump, or a pneumatic pump.

Example 11. The system of any one of examples 1 to 10, wherein the inlet multiway valve or the outlet multiway valve is: a solenoid valve, a hydraulic valve, a piezoelectric valve, or a motorized valve.

Example 12. The system of any one of examples 1 to 11 further comprising a device in fluidic connection with multiway outflow valve and configured to receive pressure from the set of two or more accumulator chambers.

Example 13. The system of example 12, wherein the device is a medical device.

Example 14. The system of example 12, wherein the device is blood pressure monitoring system.

Example 15. The system of example 14, wherein the blood pressure monitoring system comprises a pressure cuff configured to receive pressure from the set of two or more accumulator chambers.

Example 16. The system of example 14, wherein the blood pressure monitoring system utilizes a volume clamp method to measure blood pressure.

Example 17. A process for continuously providing isolating pressure from a pressure source, the process comprising:
providing a system for isolating pressure, the system comprising:
   a set of two or more accumulator chambers in fluidic connection with a pressure supply, wherein the set of two or more accumulator chambers includes a first accumulator chamber and second accumulator chamber;
   an inlet multiway valve, wherein the inlet multiway valve has an inlet port in fluidic connection with the pressure supply and an outlet port for each accumulator chamber that is in fluidic connection with its accumulator chamber; and
   an outlet multiway valve, wherein the outlet multiway valve has an inlet port for each accumulator chamber that is in fluidic connection with its accumulator chamber;
accumulating pressure from the pressure supply through the inlet multiway valve into the first accumulator chamber while simultaneously releasing pressure from the second accumulator chamber through the outlet multiway valve, wherein the inlet port of the outlet multiway valve in fluidic connection with the first accumulator chamber is closed and the outlet port of the inlet multiway valve in fluidic connection with the second accumulator chamber is closed; and
releasing pressure from the first accumulator chamber through the outlet multiway valve while simultaneously accumulating pressure from the pressure supply through the inlet multiway valve into the second accumulator chamber, wherein the inlet port of the outlet multiway valve in fluidic connection with the second accumulator chamber is closed and the outlet port of the inlet multiway valve in fluidic connection with the first accumulator chamber is closed.

Example 18. The process of example 17, wherein the accumulation into and the release of pressure from the set of two or more accumulator chambers are programmed via a timing mechanism.

Example 19. The process of example 17, wherein the accumulation into and the release of pressure from the set of two or more accumulator chambers are programmed via a pressure sensor.

Example 20. The process of example 17, 18, or 19, wherein the pressure supply is a pressure pump.

Example 21. The process of example 20, wherein the pressure pump is a positive-displacement pump, a centrifugal pump, or an axial-flow pump.

Example 22. The process of example 20, wherein the pressure pump is a rotary-type pump, a reciprocating-type pump, a linear-type pump, a piezoelectric pump, or a pneumatic pump.

Example 23. The process of any one of examples 17 to 22, wherein the inlet multiway valve or the outlet multiway valve is: a solenoid valve, a hydraulic valve, a piezoelectric valve, or a motorized valve.

Example 24. The process of any one of examples 17 to 23, wherein the pressure released from the set of two or more accumulator chambers is passed to a medical device.

Example 25. The process of example 24, wherein the device is blood pressure monitoring system.

Example 26. The process of example 25, wherein the pressure released from the set of two or more accumulator chambers is passed to a pressure cuff.

Example 27. A system for providing smoothened pressure, the system comprising:
a chamber bifurcated by a piston, yielding a first side and second side of the chamber, wherein the piston provides a seal to isolate the two sides of the chamber, and wherein the piston is powered to move back and forth within the chamber;
a first multiway valve in fluidic connection with the first side of the chamber, wherein the first multiway valve is configured to receive fluid and transfer the fluid into the first side of the chamber, and wherein the first multiway valve is further configured to release the fluid from the first side of the chamber; and
a second multiway valve in fluidic connection with the second side of the chamber, wherein the second multiway valve is configured to receive fluid and transfer the fluid into the second side of the chamber, and wherein the second multiway valve is further configured to release the fluid from the first side of the chamber.

Example 28. The system of example 27, wherein the piston is powered to move back and forth within the chamber by a motorized crank with a lever that is in connection with the piston.

Example 29. The system of example 27 or 28, wherein when the piston is powered to move away from the first side of the chamber and towards the second side chamber, the first multiway valve allows fluid to accumulate within the first side of the chamber and the second valve simultaneously allows fluid to be released from the second side of the chamber.

Example 30. The system of example 27, 28, or 29, wherein the first multiway valve and the second multiway valve are programmed to provide continuous pressure released from the chamber.

Example 31. The system of example 30, wherein the first multiway valve and the second multiway valve work in conjunction with the movement of the piston within the chamber to provide the continuous pressure.

Example 32. The system of example 30 or 31, wherein the first multiway valve and the second multiway valve are programmed via a timing mechanism.

Example 33. The system of example 30 or 31, wherein the first multiway valve and the second multiway valve are programmed via a pressure sensor.

Example 34. The system of any one of examples 27 to 33, wherein the inlet multiway valve or the outlet multiway valve is: a solenoid valve, a hydraulic valve, a piezoelectric valve, or a motorized valve.

Example 35. The system of any one of examples 27 to 34 further comprising an expansion chamber in fluidic connection with the first multiway valve or the second multiway valve and configured to receive pressure from the chamber.

Example 36. The system of example 27 further comprising a device in fluidic connection with the first multiway valve and the second multiway valve and configured to receive pressure from the chamber.

Example 37. The system of example 36, wherein the device is a medical device.

Example 38. The system of example 36, wherein the device is blood pressure monitoring system.

Example 39. The system of example 38, wherein the blood pressure monitoring system comprises a pressure cuff configured to receive pressure from the chamber.

Example 40. The system of example 38, wherein the blood pressure monitoring system utilizes a volume clamp method to measure blood pressure.

Example 41. A process for continuously providing smoothened pressure from a pressure source, the process comprising:
providing a piston system, the system comprising:
   a chamber bifurcated by a piston, yielding a first side and second side of the chamber, wherein the piston provides a seal to isolate the two sides of the chamber, and wherein the piston is powered to move back and forth within the chamber;
   a first multiway valve in fluidic connection with the first side of the chamber, wherein the first multiway valve is configured to receive fluid and transfer the fluid into the first side of the chamber, and wherein the first multiway valve is further configured to release the fluid from the first side of the chamber; and
   a second multiway valve in fluidic connection with the second side of the chamber, wherein the second multiway valve is configured to receive fluid and transfer the fluid into the second side of the chamber, and wherein the second multiway valve is further configured to release the fluid from the first side of the chamber;
accumulating fluid within the first side of the chamber via the first multiway valve while simultaneously releasing fluid from the second side of the chamber via the second multiway valve, wherein the accumulation of fluid in the first side of the chamber and the release of fluid from the second side of the chamber is induced by moving the piston away from the first side of the chamber and towards the second side of the chamber; and
releasing fluid from the first side of the chamber via the first multiway valve while simultaneously accumulating fluid within the second side of the chamber via the second multiway valve, wherein the release of fluid in the first side of the chamber and the accumulation of fluid from the second side of the chamber is induced by moving the piston toward from the first side of the chamber and away the second side of the chamber.

Example 42. The process of example 41, wherein the accumulation into and the release of fluid from the chamber via the first multiway valve and the second multiway valve are programmed via a timing mechanism.

Example 43. The process of example 41 or 42, wherein the accumulation into and the release of fluid from the chamber via the first multiway valve and the second multiway valve are programmed via a pressure sensor.

Example 44. The process of example 41, 42, or 43, wherein the first multiway valve or the second multiway valve is: a solenoid valve, a hydraulic valve, a piezoelectric valve, or a motorized valve.

Example 45. The process of any one of examples 41 to 44, wherein the pressure released from the chamber is passed to a medical device.

Example 46. The process of example 45, wherein the device is blood pressure monitoring system.

Example 47. The process of example 46, wherein the pressure released from the chamber is passed to a pressure cuff.

Example 48. A blood pressure monitoring system, comprising:
a blood pressure cuff configured to apply pressure onto an extremity of a patient and in fluidic connection for a smoothened pressure supply system, wherein the smoothened pressure supply system comprises:
   a pressure supply;
   a set of two or more accumulator chambers in fluidic connection with the pressure supply;
   an inlet multiway valve, wherein the inlet multiway valve has an inlet port in fluidic connection with the pressure supply and an outlet port for each accumulator chamber that is in fluidic connection with its accumulator chamber; and
   an outlet multiway valve, wherein the outlet multiway valve has an inlet port for each accumulator chamber that is in fluidic connection with its accumulator chamber.

Example 49. A blood pressure monitoring system, comprising:
a blood pressure cuff configured to apply pressure onto an extremity of a patient and in fluidic connection for a smoothened pressure supply system, wherein the smoothened pressure supply system comprises:
   a pressure supply;
a chamber bifurcated by a piston, yielding a first side and second side of the chamber, wherein the piston provides a seal to isolate the two sides of the chamber, and wherein the piston is powered to move back and forth within the chamber;
   a first multiway valve in fluidic connection with the first side of the chamber, wherein the first multiway valve is configured to receive fluid and transfer the fluid into the first side of the chamber, and wherein the first multiway valve is further configured to release the fluid from the first side of the chamber; and
   a second multiway valve in fluidic connection with the second side of the chamber, wherein the second multiway valve is configured to receive fluid and transfer the fluid into the second side of the chamber, and wherein the second multiway valve is further configured to release the fluid from the first side of the chamber.

1. A blood pressure monitoring system, comprising:
a blood pressure cuff configuration to apply pressure onto an extremity of a patient and in fluidic connection for a smoothened pressure supply system, wherein the smoothened pressure supply system comprises:
a pressure supply;
a set of two or more accumulator chambers in fluidic connection with the pressure supply;
an inlet multiway valve, wherein the inlet multiway valve has an inlet port in fluidic connection with the pressure supply and an outlet port for each accumulator chamber that is in fluidic connection with its accumulator chamber; and
an outlet multiway valve, wherein the outlet multiway valve has an inlet port for each accumulator chamber that is in fluidic connection with its accumulator chamber.

2. The system of claim 1, wherein the inlet multiway valve and the outlet multiway valve are programmed to sequentially alternate accumulation and release of pressure among the set of two or more accumulator chambers.

3. The system of claim 2, wherein the inlet multiway valve and the outlet multiway valve are programmed via a timing mechanism.

4. The system of claim 2, wherein the inlet multiway valve and the outlet multiway valve are programmed via a pressure sensor.

5. The system of claim 2, wherein the set of two or more accumulator chambers includes a first accumulator chamber and a second accumulator chamber, wherein the sequentially alternate accumulation and release of pressure among the set of two or more accumulator chambers comprises: when the first accumulator chamber accumulates pressure from the pressure supply via the inlet multiway valve, the second accumulator chamber concurrently releases pressure via the outlet multiway valve; and when the second accumulator chamber accumulates pressure from the pressure supply via the inlet multiway valve, the first accumulator chamber concurrently releases pressure via the outlet multiway valve.

6. The system of claim 1, wherein when a first accumulator of the set of two or more accumulator chambers receives pressure from the pressure supply, the multiway inlet valve is open and the multiway outlet valve is closed; and when the first accumulator releases pressure the multiway inlet valve is closed and the multiway outlet valve is open.

7. The system of claim 1, wherein the pressure supply comprises a pressure pump.

8. The system of claim 7, wherein the pressure pump is a positive-displacement pump, a centrifugal pump, or an axial-flow pump.

9. The system of claim 7, wherein the pressure pump is a rotary-type pump, a reciprocating-type pump, a linear-type pump, a piezoelectric pump, or a pneumatic pump.

10. The system of claim 1, wherein the inlet multiway valve or the outlet multiway valve is: a solenoid valve, a hydraulic valve, a piezoelectric valve, or a motorized valve.

11. The system of claim 1 further comprising a regulatory control for controlling an amount of pressure supplied to the blood pressure cuff.

12. The system of claim 1, wherein the blood pressure cuff comprises a pressurizable bladder configured to apply pressure onto an extremity of a patient.

13. A process for continuously providing isolating pressure from a pressure source to a blood pressure cuff, the process comprising:
providing a blood pressure monitoring system that comprises a blood pressure cuff configured to apply pressure onto an extremity of a patient and in fluidic connection for a smoothened pressure supply system, wherein the smoothened pressure supply system comprises:
a pressure supply;
a set of two or more accumulator chambers in fluidic connections with the pressure supply;
an inlet multiway valve, wherein the inlet multiway valve has an inlet port in fluidic connection with the pressure supply and an outlet port for each accumulator chamber that is in fluidic connection with its accumulator chamber; and
an outlet multiway valve, wherein the outlet multiway valve has an inlet port for each accumulator chamber that is in fluidic connection with its accumulator chamber;
accumulating pressure from the pressure supply through the inlet multiway valve into a first accumulator chamber while simultaneously releasing pressure from a second accumulator chamber through the outlet multiway valve, wherein the inlet port of the outlet multiway valve in fluidic connection with the first accumulator chamber is closed and the outlet port of the inlet multiway valve in fluidic connection with the second accumulator chamber is closed;
releasing pressure from the first accumulator chamber through the outlet multiway valve while simultaneously accumulating pressure from the pressure supply through the inlet multiway valve into the second accumulator chamber, wherein the inlet port of the outlet multiway valve in fluidic connection with the second accumulator chamber is closed and the outlet port of the inlet multiway valve in fluidic connection with the first accumulator chamber is closed; and
transferring the pressure released from the first accumulator chamber to the blood pressure cuff.

14. The process of claim 13, wherein the accumulation into and the release of pressure from the set of two or more accumulator chambers are programmed via a timing mechanism.

15. The process of claim 13, wherein the accumulation into and the release of pressure from the set of two or more accumulator chambers are programmed via a pressure sensor.

16. The process of claim 13, wherein the pressure supply comprises a pressure pump.

17. The process of claim 16, wherein the pressure pump is a positive-displacement pump, a centrifugal pump, or an axial-flow pump.

18. The process of claim 16, wherein the pressure pump is a rotary-type pump, a reciprocating-type pump, a linear-type pump, a piezoelectric pump, or a pneumatic pump.

19. The process of claim 13, wherein the inlet multiway valve or the outlet multiway valve is: a solenoid valve, a hydraulic valve, a piezoelectric valve, or a motorized valve.

20. The process of claim 13, wherein the blood pressure cuff is secured onto an extremity of a patient.
